(19) United States
(12) Patent Application Publication
(10) Pub. No.: US 2010/0331210 A1
(43) Pub. Date: Dec. 30, 2010

(54) METHODS AND SYSTEMS FOR EVALUATING THE SENSITIVITY OR RESISTANCE OF TUMOR SPECIMENS TO CHEMOTHERAPEUTIC AGENTS

(75) Inventors: Michael Gabrin, Pittsburgh, PA (US); Kui Shen, Pittsburgh, PA (US); Nan Song, Pittsburgh, PA (US); Zhenyu Ding, Pittsburgh, PA (US); David Gingrich, Pittsburgh, PA (US)

Correspondence Address: COOLEY LLP;ATTN: Patent Group, Suite 1100, 777 - 6th Street, NW, WASHINGTON, DC, 20001, US

(73) Assignee: Precision Therapeutics, Inc., Pittsburgh, PA (US)

(21) Appl. No.: 12/791,425

(22) Filed: Jun. 1, 2010

Related U.S. Application Data
(60) Provisional application No. 61/182,445, filed on May 29, 2009.

(51) Int. Cl. C40B 30/06; C12Q 1/68; C40B 40/06

(52) U.S. Cl. 506/10; 435/6; 506/16

(57) ABSTRACT

The present invention provides methods, systems, and kits for evaluating the sensitivity and/or resistance of tumor specimens to one or a combination of chemotherapeutic agents. Particularly, the invention provides malignant cell gene signatures that are predictive of a tumor's response to candidate chemotherapeutic regimens.

### FIELD OF THE INVENTION

The present invention relates to the field of molecular diagnostics, and particularly to gene expression signatures that are indicative of a tumor's sensitivity and/or resistance to therapeutic agents or combinations of agents, including chemotherapeutic agents, small molecule agents, biologics, and targeted therapies.

### BACKGROUND

Traditionally, treatments for cancer patients are selected based on agents and regimens identified to be most effective in large randomized clinical trials. However, since such therapy is not individualized, this approach often results in the administration of sub-optimal chemotherapy. The administration of sub-optimal or ineffective chemotherapy to a particular patient can lead to unsuccessful treatment, including death, disease progression, unnecessary toxicity, and higher health care costs.

In an attempt to individualize cancer treatment, in vitro drug-response assay systems (chemoresponse assays), gene expression signatures, as well as other biomarkers, have been developed to guide patient treatment decisions. However, the use of these systems are not sufficiently widespread due, in-part, to difficulties in interpreting the data in a clinically meaningful way, as may be required in many instances to drive administration of an individualized treatment regimen. For example, while in vitro systems are recognized as predicting generally inactive and/or generally active agents, and/or for predicting short-term responses, such systems are not generally recognized as providing accurate estimations of patient survival with particular treatment regimens (Fruehauf et al., Endocrine-Related Cancer 9:171-182 (2002). Further, gene expression signatures sufficient to guide patient treatment are difficult to validate, generally taking many years to identify and validate in independent patient populations. For example, identifying and validating gene expression signatures in independent patient populations generally requires access to large numbers of patient samples as well as corresponding clinical data, including the chosen course of treatment and treatment outcome.

A system that provides accurate and interpretable results with regard to a tumor's sensitivity or resistance to candidate treatments would encourage more individualized treatment plans. Such methods could present a clear advantage of an individualized treatment regimen, as compared to a non-individualized selection of agents based on large randomized trials.

### SUMMARY OF THE INVENTION

The present invention provides methods, systems, and kits for preparing gene expression profiles that are indicative of a tumor's sensitivity and/or resistance to therapeutic agents or combinations. Thus, the invention further provides methods systems, and kits for evaluating the sensitivity and/or resistance of tumor specimens to one or a combination of therapeutic agents. Particularly, the invention provides malignant cell gene expression signatures that are indicative of a tumor's sensitivity and/or resistance to candidate therapeutic regimens.

In one aspect, the invention provides methods for preparing gene expression profiles for tumor specimens and cultured cells, as well as methods for predicting a tumor's sensitivity or resistance to therapeutic agents or combinations by evaluating tumor gene expression profiles for the presence of indicative gene expression signatures. The method comprises preparing a gene expression profile for a patient tumor specimen, and evaluating the gene expression profile for the presence of one or more gene expression signatures, each gene expression signature being indicative of sensitivity or resistance to a therapeutic agent or combination of agents. By predicting the tumor's sensitivity or resistance to candidate therapeutic agents, the invention thereby provides information to guide individualized cancer treatment.

The gene expression profile may be prepared directly from patient specimens, e.g., by a process comprising RNA extraction or isolation directly from tumor specimens, or alternatively, and particularly where specimens are amenable to culture, malignant cells may be enriched (e.g., expanded) in culture for gene expression analysis. For example, malignant cells may be enriched in culture by disaggregating or mincing the tumor specimen to prepare tumor tissue explants, and allowing one or more tumor tissue explants to form a cell culture monolayer. RNA is then extracted from the cultured cells for gene expression analysis. The resulting gene expression profile, whether prepared directly from patient tumor tissue or prepared from cultured cells, contains gene transcript levels (or “expression levels”) for genes that are representative of the cells sensitivity or resistance to chemotherapeutic agents and/or combinations of agents.

The gene expression profile may be evaluated for the presence of one or more indicative gene expression signatures. For example, the profiles are compared to one or more gene expression signatures that are each indicative of sensitivity or resistance to a candidate agent or combination of agents, to thereby score or classify the patient's specimen as sensitive or resistant to such agents or combinations. The gene expression signatures in some embodiments include those generally applicable to a variety of cancer types and/or therapeutic agent(s). Alternatively, or in addition, the gene expression signatures are predictive for a particular type of cancer, such as breast cancer, and/or for a particular course of treatment. The gene signature may be predictive of survival or duration of survival, a pathological complete response (pCR) to treatment, or other measure of patient outcome, such as progression free interval or tumor size, among others.

For example, the gene expression signature may be indicative of sensitivity or resistance to one or more of cyclophosphamide, doxorubicin, fluorouracil, and paclitaxel, or the combination (e.g., “TFAC”), and exemplary gene expression signatures according to this embodiment are disclosed in Tables 1 and 2 herein. In another embodiment, the gene expression signature is indicative of sensitivity and/or resistance to treatment with one or more of cyclophosphamide and/or epirubicin (e.g., “EC” combination), and such exemplary gene expression signatures are disclosed in Tables 3 and 4 herein. Still further, the gene expression signature may be indicative of sensitivity or resistance to one or more of cyclophosphamide and/or doxorubicin (e.g., “AC” combination), and exemplary gene expression signatures according to this embodiment are disclosed in Tables 5 and 6 herein. In other embodiments, the gene signature is indicative of sensitivity or resistance to one or more of cyclophosphamide, docetaxel, and/or doxorubicin (e.g., “ACT” combination), and exemplary gene expression signatures in accordance with this embodiment are disclosed in Tables 7 and 8. Such gene expression signatures were identified in cancer cell lines by correlating the level of in vitro chemosensitivity with levels of gene expression. The resulting gene expression signatures were independently validated in patient test populations as described in detail herein.

In some embodiments, the results of gene expression analysis are combined with results from in vitro chemosensitivity testing, to provide a more complete and/or accurate prognostic and/or predictive tool for guiding patient therapy.

In a related aspect, the invention provides methods for determining gene expression signatures that are indicative of a tumor or cancer cell's sensitivity to a chemotherapeutic agent or combination. Such gene expression signatures are first identified in cancer cells by correlating the level of in vitro chemosensitivity with gene expression levels. The cultured cells may be immortalized cell lines, or may be derived directly from patient tumor specimens, for example, by enriching or expanding malignant epithelial cells from the tumor specimen in monolayer culture, and suspending the cultured cells for testing and/or RNA isolation. The resulting gene expression signatures are then independently validated in patient test populations having available gene expression data and corresponding clinical data, including information regarding the treatment regimen and outcome of treatment. This aspect of the invention reduces the length of time and quantity of patient samples needed for identifying and validating such gene expression signatures.

In other aspects, the invention provides computer systems and kits (e.g., microarray, bead set, probe set) for generating gene expression profiles that are useful for predicting a patient's response to a chemotherapeutic agent or combination, for example, in connection with the methods of the invention.

### DESCRIPTION OF THE FIGURES

FIG. 1 illustrates a method for identifying and validating gene expression signatures. Cancer cell lines are used for determining gene expression levels, as well as levels of in vitro sensitivity/resistance to chemotherapeutics agents or combinations of agents. Gene expression signatures indicative of resistance and/or sensitivity to these agents or combinations in vitro are identified by correlating in vitro responses with gene expression levels. The resulting gene expression signature(s) are validated in a patient population by evaluating patient tumor gene expression data for the presence of the gene expression signatures. Patient samples are scored and/or classified as resistant and/or sensitive to chemotherapeutic agents on the basis of the gene signatures, thereby obtaining an outcome prediction. The accuracy of the classification or prediction is tested by comparing the prediction with the actual outcome of treatment.

FIG. 2 illustrates the accuracy of a 423-gene signature from Tables 1 and 2 for predicting pCR in an independent patient population (133 neoadjuvant breast cancer patients treated with TFAC). Outcome is pathological complete response (pCR). The results are shown as a receiver operator curve (ROC).

FIG. 3 illustrates the accuracy of a 370-gene signature from Tables 3 and 4 for predicting pCR in an independent patient population (37 neoadjuvant breast cancer patients treated with EC). Outcome is pathological complete response (pCR). The results are shown as a receiver operator curve (ROC).

FIG. 4 illustrates the accuracy of a 371-gene signature from Tables 5 and 6 for predicting pCR in an independent patient population (326 neoadjuvant breast cancer patients treated with AC or ACT). Outcome is pathological complete response (pCR). The results are shown as a receiver operator curve (ROC).

FIG. 5 illustrates the accuracy of a 402-gene signature from Tables 7 and 8 for predicting pCR in an independent patient population (326 neoadjuvant breast cancer patients treated with AC or ACT). Outcome is pathological complete response (pCR). The results are shown as a receiver operator curve (ROC).

FIG. 6 illustrates the accuracy of the AC-trained signature of Tables 5 and 6 for predicting pCR upon treatment with ACT (left panel), and the accuracy of the ACT-trained signature of Tables 6 and 7 for predicting pCR upon treatment with AC (right panel). Results are shown as ROCs.

FIG. 7 illustrates that the multigene predictors are stable. The left panel shows that the gene expression signature of Tables 1 and 2 is stable over a large range of increasing gene number, from less than about 10 to over 400 genes. The right panel shows that the gene expression signature of Tables 3 and 4 is stable over a large range of increasing gene number, from about 100 to over about 400 genes.

FIG. 8 illustrates that the multigene predictors are stable. The left panel shows that the gene expression signature of Tables 5 and 6 are stable over a large range of increasing gene number, from less than about 10 to over 400 genes. The right panel shows that the gene expression signature of Tables 7 and 8 are stable over a large range of increasing gene number, from about 10 up to about 400 genes.

FIG. 9 summarizes the results for four exemplary multigene predictors.

### DETAILED DESCRIPTION OF THE INVENTION

The present invention provides methods, systems, and kits for preparing gene expression profiles that are indicative of a tumor's sensitivity and/or resistance to therapeutic agents or combinations. Thus, the invention further provides methods systems, and kits for evaluating the sensitivity and/or resistance of tumor specimens to one or a combination of therapeutic agents. The invention provides malignant cell, gene expression signatures that are indicative of a tumor's sensitivity and/or resistance to candidate chemotherapeutic regimens.

### Methods for Gene Profiling and Predicting Response to Treatment

The invention provides methods for preparing gene expression profiles for tumor specimens, as well as methods for evaluating a tumor's sensitivity and/or resistance to one or more therapeutic agents or combinations of agents. For example, the gene expression profile generated for a tumor specimen, or cultured cells derived therefrom, is evaluated for the presence of one or more indicative gene expression signatures. The gene expression signatures are indicative of a response to a treatment regimen. In this aspect, the invention provides information to guide a physician in designing/administering an individualized therapeutic regimen for a cancer patient.

The patient generally is one with a cancer or neoplastic condition, such as one that is treated with the therapeutic agents described herein. The patient may suffer from cancer of essentially any tissue or organ, including but not limited to breast, ovaries, lung, colon, skin, prostate, kidney, endometrium, nasopharynx, pancreas, head and neck, kidney, and brain, among others. The patient may be inflicted with a carcinoma or sarcoma. The patient may have a solid tumor of epithelial origin. The tumor specimen may be obtained from the patient by surgery, or may be obtained by biopsy, such as a fine needle biopsy or other procedure prior to the selection/initiation of therapy. In certain embodiments, the cancer is breast cancer, including preoperative or post-operative breast cancer. In certain embodiments, the patient has not undergone treatment to remove the breast tumor, and therefore is a candidate for neoadjuvant therapy.

The cancer may be primary or recurrent, and may be of any type (as described above), stage (e.g., Stage I, II, III, or IV or an equivalent of other staging system), and/or histology (e.g., serous adenocarcinoma, endometroid adenocarcinoma, mucinous adenocarcinoma, undifferentiated adenocarcinoma, transitional cell adenocarcinoma, or adenocarcinoma, etc.). The patient may be of any age, sex, performance status, and/or extent and duration of remission.

In certain embodiments, the patient is a candidate for treatment with one or more of cyclophosphamide, doxorubicin, fluorouracil, and paclitaxel, or the combination (e.g., “TFAC”). In other embodiments, the patient is a candidate for treatment with one or more of cyclophosphamide and/or epirubicin (e.g., “EC” combination). Still further, the patient may be a candidate for treatment with one or more of cyclophosphamide and/or doxorubicin (e.g., “AC” combination). In other embodiments, the patient is a candidate for treatment with one or more of cyclophosphamide, docetaxel, and/or doxorubicin (e.g., “ACT” combination).

The gene expression profile is determined for a tumor tissue or cell sample, such as a tumor sample removed from the patient by surgery or biopsy. The tumor sample may be “fresh,” in that it was removed from the patent within about five days of processing, and remains suitable or amenable to culture. In some embodiments, the tumor sample is not “fresh,” in that the sample is not suitable or amenable to culture. Tumor samples are generally not fresh after from 3 to 7 days (e.g., about five days) of removal from the patient. The sample may be frozen after removal from the patient, and preserved for later RNA isolation. The sample for RNA isolation may be a formalin-fixed paraffin-embedded (FFPE) tissue.

In certain embodiments, the malignant cells are enriched or expanded in culture by forming a monolayer culture from tumor sample explants. For example, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments, such as for ovarian or colorectal tumors.

For example, where it is desirable to expand and/or enrich malignant cells in culture relative to non-malignant cells that reside in the tumor, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³. After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of days to a few weeks a cell monolayer will form.

Further, it is believed that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explant may be agitated to substantially loosen or release tumor cells from the tumor explant, and the released cells cultured to produce a cell culture monolayer. The use of this procedure to form a cell culture monolayer helps maximize the growth of representative malignant cells from the tissue sample. Monolayer growth rate and/or cellular morphology (e.g., epithelial character) may be monitored using, for example, a phase-contrast inverted microscope. Generally, the cells of the monolayer are actively growing at the time the cells are suspended for RNA extraction.

The process for enriching or expanding malignant cells in culture is described in U.S. Pat. Nos. 5,728,541, 6,900,027, 6,887,680, 6,933,129, 6,416,967, 7,112,415, 7,314,731, and 7,501,260 (all of which are hereby incorporated by reference in their entireties). The process may further employ the variations described in US Published Patent Application Nos. 2007/0059821 and 2008/0085519, both of which are hereby incorporated by reference in their entireties.

In preparing the gene expression profile, RNA is extracted from the tumor tissue or cultured cells by any known method. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, there are various products commercially available for RNA isolation which may be used. Total RNA or polyA+ RNA may be used for preparing gene expression profiles in accordance with the invention.

The gene expression profile is then generated for the samples using any of various techniques known in the art, and described in detail elsewhere herein. Such methods generally include, without limitation, hybridization-based assays, such as microarray analysis and similar formats (e.g., Whole Genome DASL™ Assay, Illumina, Inc.), polymerase-based assays, such as RT-PCR (e.g., Tagman™), flap-endonuclease-based assays (e.g., Invader™), as well as direct mRNA capture with branched DNA (QuantiGene™) or Hybrid Capture™ (Digene).

The gene expression profile contains gene expression levels for a plurality of genes whose expression levels are predictive or indicative of the tumor's response to one or a combination of therapeutic agents. Such genes are listed collectively in Tables 1-8. As used herein, the term “gene,” refers to a DNA sequence expressed in a sample as an RNA transcript, and may be a full-length gene (protein encoding or non-encoding) or an expressed portion thereof such as expressed sequence tag or “EST.” Thus, the genes listed in Tables 1-8 are each independently a full-length gene sequence, whose expression product is present in samples, or is a portion of an expressed sequence detectable in samples, such as an EST sequence.

The genes listed in Tables 1-8 may be differentially expressed in drug-sensitive samples versus ⁻drug-resistant samples as described below. As used herein, “differentially expressed” means that the level or abundance of an RNA transcript (or abundance of an RNA population sharing a common target (or probe-hybridizing) sequence, such as a group of splice variant RNAs) is significantly higher or lower in a drug-sensitive sample as compared to a reference level (e.g., a drug resistant sample). For example, the level of the RNA or RNA population may be higher or lower than a reference level. The reference level may be the level of the same RNA or RNA population in a control sample or control population (e.g., a Mean level for a drug-resistant sample), or may represent a cut-off or threshold level for a sensitive or resistant designation.

The gene expression profile generally contains the expression levels for at least about 10, 25, 50, 100, 200, 400, or at least about 500 genes listed collectively in Tables 1-8. As discussed, these expression levels represent the gene expression state of the patient's malignant cells or tumor, and are evaluated for the presence of one or more gene signatures indicative of the tumor's sensitivity and/or resistance to chemotherapeutic agents. In such embodiments, the gene expression profile contains the level of expression of 3000 genes or less, 2000 genes or less, 1000 genes or less, or 500 genes or less, such as to be prepared with the use of a custom microarray or probe set.

Tables 1, 3, 5, and 7 each list raw gene expression levels (e.g., no log2 transformation) for each cell line in a collection of breast cancer cell lines. Neve, R M, Chin, K, Fridlyand, J, Yeh, J, Baehner, F L, Fevr, T, Clark, L, Bayani, N, Coppe, J P, Tong, F, Speed, T, Spellman, P T, DeVries, S, Lapuk, A, Wang, N J, Kuo, W L, Stilwell, J L, Pinkel, D, Albertson, D G, Waldman, F M, McCormick, F, Dickson, R B, Johnson, M D, Lippman, M, Ethier, S, Gazdar, A, Gray, J W (2006). A collection of breast cancer cell lines for the study of functionally distinct cancer subtypes. Cancer Cell, 10, 6:515-27. Gene expression data for the cell lines determined with the hgu133a microarray platform (Affymetrix) is publicly available. Each of Tables 1 a-1 h, 3a-3h, 5a-5h, and 7a-7h each provides the gene expression data for 2 or 3 cell lines in the collection. Cell lines are listed across the top header row, with probe ID and corresponding gene listed in the left-hand columns.

Table 1 lists genes that are expressed at significantly different levels in TFAC-sensitive and TFAC-resistant cell lines. TFAC refers to the combination cyclophosphamide, doxorubicin, fluorouracil, and paclitaxel. Table 3 lists genes that are expressed at significantly different levels in EC-sensitive versus EC-resistant cell lines. EC refers to the combination cyclophosphamide and doxorubicin. Table 5 lists genes that are expressed at significantly different levels in AC-sensitive versus AC-resistant cell lines. AC refers to the combination of cyclophosphamide and/or doxorubicin. Table 7 lists genes that are expressed at significantly different levels in ACT-sensitive versus ACT-resistant cell lines. ACT refers to the combination cyclophosphamide, docetaxel, and doxorubicin. Sequences that correspond to these genes are known, and the publicly available sequences are hereby incorporated by reference.

Tables 2, 4, 6, and 8 list the mean expression scores for cell lines that are resistant and sensitive to TFAC, EC, AC, and ACT, respectively. Tables 2, 4, 6, and 8 include the sensitive and resistant mean expression scores for each gene, and list the fold change from sensitive to resistant. For example, where x is the mean expression score for sensitive cell lines for a particular gene, and y is the mean expression score for resistant cell lines for that gene, fold change is represented by mean X/mean Y. Sensitivity and resistance to the indicated drug or combination were determined for each cell line in vitro as an AUC value essentially as described herein, and the top ⅓ values were designated as sensitive, and the bottom ⅓ values were designated as resistant.

Tables 9-12 list, along with the probe ID and Gene Symbol for each of the signatures of Tables 1, 3, 5, and 7 (respectively), an Entrez ID for each gene and a weight score for its association with drug-sensitivity. The Probe and gene sequences listed are publicly available, and are hereby incorporated by reference.

Thus, in accordance with this aspect, the gene expression profile, which is generated from the tumor specimen or malignant cells cultured therefrom as described, may contain the levels of expression for at least about 3 genes listed in Tables 1 and 2. In some embodiments, the patient's gene expression profile contains the levels of expression for at least about 5, 7, 10, 12, 15, 20, 25, 40, 50, 75, 100, or 200 genes listed in Tables 1 and 2, such genes being differentially expressed in drug-sensitive tumor cells (e.g., TFAC-sensitive cells) versus drug resistant tumor cells, and which may be breast cancer cells. In some embodiments, the gene expression profile may contain the levels of expression for all or substantially all genes listed in Tables 1 and 2, such as at least about 250, 300, or 400 genes.

Alternatively or in addition, the gene expression profile may contain the levels of expression for at least about 3 genes listed in Tables 3 and 4. In some embodiments, the patient's gene expression profile contains the levels of expression for at least about 5, 7, 10, 12, 15, 20, 25, 40, 50, 75, 100, or 200 genes listed in Tables 3 and 4, such genes being differentially expressed in drug-sensitive tumor cells (e.g., EC-sensitive cells) versus drug resistant tumor cells, and which may be breast cancer cells. In some embodiments, the gene expression profile may contain the levels of expression for all or substantially all genes listed in Tables 3 and 4, such as at least about 250, 300 or 400 genes.

Alternatively or in addition, the gene expression profile may contain the levels of expression for at least about 3 genes listed in Tables 5 and 6. In some embodiments, the patient's gene expression profile contains the levels of expression for at least about 5, 7, 10, 12, 15, 20, 25, 40, 50, 75, 100, or 200 genes listed in Tables 5 and 6, such genes being differentially expressed in drug-sensitive tumor cells (e.g., AC-sensitive cells) versus drug resistant tumor cells, and which may be breast cancer cells. In some embodiments, the gene expression profile may contain the levels of expression for all or substantially all genes listed in Tables 5 and 6, such as at least about 250, 300, or 400 genes.

Alternatively or in addition, the gene expression profile may contain the levels of expression for at least about 3 genes listed in Tables 7 and 8. In some embodiments, the patient's gene expression profile contains the levels of expression for at least about 5, 7, 10, 12, 15, 20, 25, 40, 50, 75, 100, or 200 genes listed in Tables 7 and 8, such genes being differentially expressed in drug-sensitive tumor cells (e.g., ACT-sensitive cells) versus drug resistant tumor cells, and which may be breast cancer cells. In some embodiments, the gene expression profile may contain the levels of expression for all or substantially all genes listed in Tables 7 and 8, such as at least about 250, 300, or 400 genes.

In certain embodiments, the gene expression profile contains a measure of expression levels for a plurality of genes (e.g., 5, 7, 10, 12, 15, 50, etc.) that are each, independently, expressed in drug-sensitive versus drug-resistant samples by a fold change magnitude (up or down) of at least about 1.2 (up) or about 0.8 (down). As discussed previously, fold change magnitude is defined as mean sensitive score/mean resistant score. In some embodiments, the plurality of genes are differentially expressed in drug sensitive versus drug resistant cells by a fold change magnitude (up) of at least 1.5, or at least about 1.7, or at least about 2, or at least about 2.5, or by a fold magnitude (down) of less than about 0.7, about 0.5, or about 0.4. Alternatively, the expression levels (mean sensitive and mean resistant) may differ by at least about 2-, 3-, 4-, or 5-, 10-fold, or more. Tables 2, 4, 6, and 8 list genes by differential levels of expression in drug-sensitive versus drug-resistant cells, respectively, and such levels may be used to select genes for profiling in accordance with this paragraph.

The gene expression profile prepared according to this aspect of the invention is evaluated for the presence of one or more drug-sensitive and/or drug-resistant signatures. The gene expression signature(s) comprise the gene expression levels indicative of a drug-sensitive and/or drug-resistant cell, so as to enable a classification of the tumor's profile as sensitive or resistant. Specifically, the gene expression signature comprises indicative gene expression levels for a plurality of genes listed in one or more of Tables 1-8, such as at least 5, 7, 10, 12, 15, 20, 25, 40, 50, 75, 100, 200, 250, 300, 400, or 500 genes listed in one or more of Tables 1-8. The signature may comprise the Mean expression levels listed in Tables 2, 4, 6, and/or 8, or alternatively, may be prepared from other data sets or using other statistical criteria.

The gene expression signature(s) may be in a format consistent with any nucleic acid detection format, such as those described herein, and will generally be comparable to the format used for profiling patient samples. For example, the gene expression signature and patient profiles may both be prepared by nucleic acid hybridization method, and with the same hybridization platform and controls so as to facilitate comparisons. The gene expression signatures may further embody any number of statistical measures to distinguish drug-sensitive and/or drug-resistant levels, including Mean expression levels and/or cut-off or threshold values. Such signatures may be prepared from the data sets disclosed herein or independent gene expression data sets.

Once the gene expression profile for patient samples are prepared, the profile is evaluated for the presence of one or more of the gene signatures, by scoring or classifying the patient profile against each gene signature.

Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

Generally, the gene expression profiles for patient specimens are scored or classified as drug-sensitive signatures or drug-resistant signatures, including with stratified or continuous intermediate classifications or scores reflective of drug sensitivity. As discussed, such signatures may be assembled from gene expression data disclosed herein (Tables 1-8), or prepared from independent data sets. The signatures may be stored in a database and correlated to patient tumor gene expression profiles in response to user inputs.

After comparing the patient's gene expression profile to the drug-sensitive and/or drug-resistant signature, the sample is classified as, or for example, given a probability of being, a drug-sensitive profile or a drug-resistant profile. The classification may be determined computationally based upon known methods as described above. The result of the computation may be displayed on a computer screen or presented in a tangible form, for example, as a probability (e.g., from 0 to 100%) of the patient responding to a given treatment. The report will aid a physician in selecting a course of treatment for the cancer patient. For example, in certain embodiments of the invention, the patient's gene expression profile will be determined to be a drug-sensitive profile on the basis of a probability, and the patient will be subsequently treated with that drug or combination. In other embodiments, the patient's profile will be determined to be a drug-resistant profile, thereby allowing the physician to exclude that candidate treatment for the patient, thereby sparing the patient the unnecessary toxicity.

In various embodiments, the method according to this aspect of the invention distinguishes a drug-sensitive tumor from a drug-resistant tumor with at least about 60%, 75%, 80%, 85%, 90% or greater accuracy (e.g., sensitivity and/or specificity). In this respect, the method according to this aspect may lend additional or alternative predictive value over standard methods, such as for example, gene expression tests known in the art, or chemoresponse testing.

The methods of the invention aid the prediction of an outcome of treatment. That is, the gene expression signatures are each predictive of an outcome upon treatment with a candidate agent or combination. The outcome may be quantified in a number of ways. For example, the outcome may be an objective response, a clinical response, or a pathological response to a candidate treatment. The outcome may be determined based upon the techniques for evaluating response to treatment of solid tumors as described in Therasse et al., New Guidelines to Evaluate the Response to Treatment in Solid Tumors, J. of the National Cancer Institute 92(3):205-207 (2000), which is hereby incorporated by reference in its entirety. For example, the outcome may be survival (including overall survival or the duration of survival), progression-free interval, or survival after recurrence. The timing or duration of such events may be determined from about the time of diagnosis or from about the time treatment (e.g., chemotherapy) is initiated. Alternatively, the outcome may be based upon a reduction in tumor size, tumor volume, or tumor metabolism, or based upon overall tumor burden, or based upon levels of serum markers especially where elevated in the disease state (e.g., PSA). The outcome in some embodiments may be characterized as a complete response, a partial response, stable disease, and progressive disease, as these terms are understood in the art.

In certain embodiments, the gene signature is indicative of a pathological complete response upon treatment with a particular candidate agent or combination (as already described). A pathological complete response, e.g., as determined by a pathologist following examination of tissue (e.g., breast or nodes in the case of breast cancer) removed at the time of surgery, generally refers to an absence of histological evidence of invasive tumor cells in the surgical specimen.

### Chemoresponse Assay

The present invention may further comprise conducting chemoresponse testing with a panel of chemotherapeutic agents on cultured cells from a cancer patient, to thereby add additional predictive value. That is, the presence of one or more gene expression signatures in tumor cells, and the in vitro chemoresponse results for the tumor specimen, are used to predict an outcome of treatment (e.g., survival, pCR, etc.). For example, where the gene expression profile and chemoresponse test both indicate that a tumor is sensitive or resistant to a particular treatment, the predictive value of the method may be particularly high.

In other aspects of the invention, in vitro chemoresponse testing is used for identifying gene signatures in cultured malignant cells (e.g., immortalized cell lines or cultures derived directly from patient cells), as described elsewhere herein. For example, the identification of gene expression signatures within tumor gene expression profiles (the signatures being indicative of sensitivity and/or resistance to treatment regimens) may be supervised using results obtained from the in vitro chemoresponse test described herein.

Several in vitro chemoresponse systems are known and art, and some are reviewed in Fruehauf et al., In vitro assay-assisted treatment selection for women with breast or ovarian cancer, Endocrine-Related Cancer 9: 171-82 (2002). In certain embodiments, the chemoresponse assay is as described in U.S. Pat. Nos. 5,728,541, 6,900,027, 6,887,680, 6,933,129, 6,416,967, 7,112,415, 7,314,731, 7,501,260 (all of which are hereby incorporated by reference in their entireties). The chemoresponse method may further employ the variations described in US Published Patent Application Nos. 2007/0059821 and 2008/0085519, both of which are hereby incorporated by reference in their entireties.

Briefly, in certain embodiments, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³.

After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of days to a few weeks a cell monolayer will form. Further, it is believed (without any intention of being bound by the theory) that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explant may be agitated to substantially release tumor cells from the tumor explant, and the released cells cultured to produce a cell culture monolayer. The use of this procedure to form a cell culture monolayer helps maximize the growth of representative tumor cells from the tissue sample.

Prior to the chemotherapy assay, the growth of the cells may be monitored, and data from periodic counting may be used to determine growth rates which may or may not be considered parallel to growth rates of the same cells in vivo in the patient. If growth rate cycles can be documented, for example, then dosing of certain active agents can be customized for the patient. Monolayer growth rate and/or cellular morphology may be monitored using, for example, a phase-contrast inverted microscope. Generally, the cells of the monolayer should be actively growing at the time the cells are suspended and plated for drug exposure. The epithelial character of the cells may be confirmed by any number of methods. Thus, the monolayers will generally be non-confluent monolayers at the time the cells are suspended for drug exposure.

A panel of active agents may then be screened using the cultured cells. Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Each test well is then contacted with at least one pharmaceutical agent, for example, an agent for which a gene expression signature is available. Such agents include cyclophosphamide, doxorubicin, fluorouracil, and paclitaxel, or the combination (e.g., “TFAC”), cyclophosphamide and/or epirubicin (e.g., “EC” combination), one or more of cyclophosphamide and/or doxorubicin (e.g., “AC” combination), and one or more of cyclophosphamide, docetaxel, and/or doxorubicin (e.g., “ACT” combination).

Alternatively, suitable pharmaceutical agents for training gene signatures by in vitro chemoresponse include small molecule agents, biologics, and targeted therapies. Exemplary agents are listed in the following table.

| Drug Name | Alternative Nomenclature |
|---|---|
| Altretamine | Hexalen ®, hydroxymethylpentamethylmelamine (HMPMM) |
| Bleomycin | Blenoxane ® |
| Carboplatin | Paraplatin ® |
| Carmustine | BCNU, BiCNU ® |
| Cisplatin | Platinol ®, CDDP |
| Cyclophosphamide | Cytoxan ®, Neosar ®, 4-hydroperoxycyclophosphamide, 4-HC |
| Docetaxel | Taxotere ®, D-Tax |
| Doxorubicin | Adriamycin ®, Rubex ®, Doxil ®* |
| Epirubicin | Ellence ® |
| Erlotinib | Tarceva ® , OSI-774 |
| Etoposide | VePesid ®, Etopophos ®, VP-16 |
| Fluorouracil | Adrucil ®, 5-FU, Efudex ®, Fluoroplex ®, Capecitabine*, Xeloda ®* |
| Gemcitabine | Gemzar ® |
| Ifosfamide | Ifex ®, 4-hydroperoxyifosfamide, 4-HI |
| Irinotecan/SN-38 | Camptosar ®, CPT-11, SN-38 |
| Leucovorin | Wellcovorin ® |
| Lomustine | CCNU, CeeNU ® |
| Melphalan | Alkeran ®, L-PAM |
| Mitomycin | Mutamycin ®, Mitozytrex ®, Mitomycin-C |
| Oxaliplatin | Eloxatin ® |
| Paclitaxel | Taxol ®, Abraxane ®* |
| Procarbazine | Matulane ®, PCZ |
| Temozolomide | Temodar ® |
| Topotecan | Hycamtin ® |
| Vinblastine | Velban ®, Exal ®, Velbe ®, Velsar ®, VLB |
| Vincristine | Oncovin ®, Vincasar PFS ®, VCR |
| Vinorelbine | Navelbine ®, NVB |

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment. The cells may be imaged once or multiple times, prior to or during contact with each treatment. Of course, any method for determining the viability of the cells may be used to assess the efficacy of each treatment in vitro.

In this manner the in vitro efficacy grade for each agent in the panel may be determined. While any grading system may be employed (including continuous or stratified), in certain embodiments the grading system is stratified, having from 2 or 3, to 10 response levels, e.g., about 3, 4, or 5 response levels. For example, when using three levels, the three grades may correspond to a responsive grade (e.g., sensitive), an intermediate responsive grade, and a non-responsive grade (e.g., resistant), as discussed more fully herein. In certain embodiments, the patient's cells show a heterogeneous response across the panel of agents, making the selection of an agent particularly crucial for the patient's treatment.

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC).

However, since a dose-response curve only reflects the cell survival pattern in the presence of a certain tested drug, assays for different drugs and/or different cell types have their own specific cell survival pattern. Thus, dose response curves that share the same aAUC value may represent different drug effects on cell survival. Additional information may therefore be incorporated into the scoring of the assay. In particular, a factor or variable for a particular drug or drug class (such as those drugs and drug classes described) and/or reference scores may be incorporated into the algorithm.

For example, in certain embodiments, the invention quantifies and/or compares the in vitro sensitivity/resistance of cells to drugs having varying mechanisms of action, and thus, in some cases, different dose-response curve shapes. In these embodiments, the invention compares the sensitivity of the patient's cultured cells to a plurality of agents that show some effect on the patient's cells in vitro (e.g., all score sensitive to some degree), so that the most effective agent may be selected for therapy. In such embodiments, an aAUC (or “weighted response score”) is calculated to take into account the shape of a dose response curve for any particular drug or drug class. The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxicity.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}−CI_{d-1})/Unit of Dose, or S_{d}=(Cl_{d-1}/Unit of Dose.

Step 3: Calculate a slope weight at each dose point, e.g., W_{d}=1−S_{d}.

Step 4: Compute aAUC, where aAUC=ΣW_{d} CI_{d}, and where, d=1, 2, . . . , 10; aAUC˜(0, 10); And at d=1, then CI_{d-1}=1. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., about 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells. For example, a plurality of drugs may be tested, and aAUC determined as above for each, to determine whether the patient's cells have a sensitive response, intermediate response, or resistant response to each drug.

In some embodiments, each drug is designated as, for example, sensitive, or resistant, or intermediate, by comparing the aAUC test value to one or more cut-off values for the particular drug (e.g., representing sensitive, resistant, and/or intermediate aAUC scores for that drug). The cut-off values for any particular drug may be set or determined in a variety of ways, for example, by determining the distribution of a clinical outcome within a range of corresponding aAUC reference scores. That is, a number of patient tumor specimens are tested for chemosenstivity/resistance (as described herein) to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures.

In other embodiments, the aAUC scores may be adjusted for drug or drug class. For example, aAUC values for dose response curves may be regressed over a reference scoring algorithm adjusted for test drugs. The reference scoring algorithm may provide a categorical outcome, for example, sensitive (s), intermediate sensitive (i) and resistant (r), as already described. Logistic regression may be used to incorporate the different information, i.e., three outcome categories, into the scoring algorithm. However, regression can be extended to other forms, such as linear or generalized linear regression, depending on reference outcomes. The regression model may be fitted as the following: Logit(Pref)=α+β(aAUC)+γ(drugs), where γ is a covariate vector and the vector can be extended to clinical and genomic features. The score may be calculated as Score=β(aAUC)+γ(drugs). Since the score is a continuous variable, results may be classified into clinically relevant categories, i.e., sensitive (S), intermediate sensitive (I), and resistant (R), based on the distribution of a reference scoring category or maximized sensitivity and specificity relative to the reference.

As stated, the chemoresponse -score for cultures derived from patient specimens may provide additional predictive or prognostic value in connection with the gene expression profile analysis.

Alternatively, where applied to immortalized cell line collections or patient-derived cultures, the in vitro chemoresponse assay may be used to supervise or train gene expression signatures. Once gene expression signatures are identified in cultured cells, e.g., by correlating the level of in vitro chemosensitivity with gene expression levels, the resulting gene expression signatures may be independently validated in patient test populations having available gene expression data and corresponding clinical data, including information regarding the treatment regimen and outcome of treatment. This aspect of the invention reduces the length of time and quantity of patient samples needed for identifying and validating such gene expression signatures.

### Gene Expression Assay Formats

Gene expression profiles, including patient gene expression profiles and the drug-sensitive and drug-resistant signatures as described herein, may be prepared according to any suitable method for measuring gene expression. That is, the profiles may be prepared using any quantitative or semi-quantitative method for determining RNA transcript levels in samples. Such methods include polymerase-based assays, such as RT-PCR, Taqman™, hybridization-based assays, for example using DNA microarrays or other solid support (e.g., Whole Genome DASL™ Assay, Illumine, Inc.), nucleic acid sequence based amplification (NASBA), flap endonuclease-based assays, as well as direct mRNA capture with branched DNA (QuantiGene™) or Hybrid Capture™ (Digene). The assay format, in addition to determining the gene expression levels for a combination of genes listed in one or more of Tables 1-8, will also allow for the control of, inter alia, intrinsic signal intensity variation between tests. Such controls may include, for example, controls for background signal intensity and/or sample processing, and/or other desirable controls for gene expression quantification across samples. For example, expression levels between samples may be controlled by testing for the expression level of one or more genes that are not differentially expressed between drug-sensitive and drug-resistant cells, or which are generally expressed at similar levels across the population. Such genes may include constitutively expressed genes, many of which are known in the art. Exemplary assay formats for determining gene expression levels, and thus for preparing gene expression profiles and drug-sensitive and drug-resistant signatures are described in this section.

The nucleic acid sample is typically in the form of mRNA or reverse transcribed mRNA (cDNA) isolated from a tumor tissue sample or a derived cultured cell population. In some embodiments, the nucleic acids in the sample may be cloned or amplified, generally in a manner that does not bias the representation of the transcripts within a sample. In some embodiments, it may be preferable to use total RNA or polyA+ RNA as a source without cloning or amplification, to avoid additional processing steps.

As is apparent to one of skill in the art, nucleic acid samples used in the methods of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or specimen of interest. Such samples also include DNA amplified from the cDNA, and RNA transcribed from the amplified DNA.

In determining a tumor's gene expression profile, or in determining a drug-sensitive or drug-resistant profile in accordance with the invention, a hybridization-based assay may be employed. Nucleic acid hybridization involves contacting a probe and a target sample under conditions where the probe and its complementary target sequence (if present) in the sample can form stable hybrid duplexes through complementary base pairing. The nucleic acids that do not form hybrid duplexes may be washed away leaving the hybridized nucleic acids to be detected, typically through detection of an attached detectable label. It is generally recognized that nucleic acids may be denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In certain embodiments, hybridization is performed at low stringency, such as 6×SSPET at 37° C. (0.005% Triton X-100), to ensure hybridization, and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that may be present, as described below (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

The hybridized nucleic acids are typically detected by detecting one or more labels attached to the sample nucleic acids. The labels may be incorporated by any of a number of means well known to those of skill in the art. See WO 99/32660.

Numerous hybridization assay formats are known, and which may be used in accordance with the invention. Such hybridization-based formats include solution-based and solid support-based assay formats. Solid supports containing oligonucleotide probes designed to detect differentially expressed genes (e.g., listed in Tables 1-8) can be filters, polyvinyl chloride dishes, particles, beads, microparticles or silicon or glass based chips, etc. Any solid surface to which oligonucleotides can be bound, either directly or indirectly, either covalently or non-covalently, may be used. Bead-based assays are described, for example, in U.S. Pat. Nos. 6,355,431, 6,396,995, and 6,429,027, which are hereby incorporated by reference. Other chip-based assays are described in U.S. Pat. Nos. 6,673,579, 6,733,977, and 6,576,424, which are hereby incorporated by reference.

An exemplary solid support is a high density array or DNA chip, which may contain a particular oligonucleotide probes at predetermined locations on the array. Each predetermined location may contain more than one molecule of the probe, but each molecule within the predetermined location has an identical probe sequence. Such predetermined locations are termed features. Probes corresponding to the genes of Tables 1-8 may be attached to single or multiple solid support structures, e.g., the probes may be attached to a single chip or to multiple chips to comprise a chip set. An exemplary chip format is hgu133a (Affymetrix).

Oligonucleotide probe arrays for determining gene expression can be made and used according to any techniques known in the art (see for example, Lockhart et al (1996), Nat Biotechnol 14:1675-1680; McGall et al. (1996), Proc Nat Acad Sci USA 93:13555-13460). Such probe arrays may contain the oligonucleotide probes necessary for determining a tumor's gene expression profile, or for preparing drug-resistant and drug-sensitive signatures. Thus, such arrays may contain oligonucleotide designed to hybridize to at least 2, 3, 4, 5, 6, 7, 8, 9, 10, 20, 30, 50, 70, 100, 200, 300 or more of the genes described herein (e.g., as described in one of Tables 1-8, or as described in any of Tables 1-8). In some embodiments, the array contains probes designed to hybridize to all or nearly all of the genes listed in one or more of Tables 1-8. In still other embodiments, arrays are constructed that contain oligonucleotides designed to detect all or nearly all of the genes in Tables 1-8 on a single solid support substrate, such as a chip or a set of beads. The array, bead set, or probe set may contain, in some embodiments, no more than 3000 probes, no more than 2000 probes, no more than 1000 probes, or no more than 500 probes, so as to embody a custom probe set for determining gene expression signatures in accordance with the invention.

Probes based on the sequences of the genes described herein for preparing expression profiles may be prepared by any suitable method. Oligonucleotide probes, for hybridization-based assays, will be of sufficient length or composition (including nucleotide analogs) to specifically hybridize only to appropriate, complementary nucleic acids (e.g., exactly or substantially complementary RNA transcripts or cDNA). Typically the oligonucleotide probes will be at least about 10, 12, 14, 16, 18, 20 or 25 nucleotides in length. In some cases, longer probes of at least 30, 40, or 50 nucleotides may be desirable. In some embodiments, complementary hybridization between a probe nucleic acid and a target nucleic acid embraces minor mismatches (e.g., one, two, or three mismatches) that can be accommodated by reducing the stringency of the hybridization media to achieve the desired detection of the target polynucleotide sequence. Of course, the probes may be perfect matches with the intended target probe sequence, for example, the probes may each have a probe sequence that is perfectly complementary to a target sequence (e.g., a sequence of a gene listed in Tables 1-8).

A probe is a nucleic acid capable of binding to a target nucleic acid of complementary sequence through one or more types of chemical bonds, usually through complementary base pairing, usually through hydrogen bond formation. A probe may include natural (i.e., A, G, U, C, or T) or modified bases (7-deazaguanosine, inosine, etc.), or locked nucleic acid (LNA). In addition, the nucleotide bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages.

When using hybridization-based assays, in may be necessary to control for background signals. The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each location of the array. In an exemplary embodiment, background is calculated as the average hybridization signal intensity for the lowest 5% to 10% of the probes in the array. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all. Of course, one of skill in the art will appreciate that hybridization signals may be controlled for background using one or a combination of known approached, including one or a combination of approaches described in this paragraph.

The hybridization-based assay will be generally conducted under conditions in which the probe(s) will hybridize to their intended target subsequence, but with only insubstantial hybridization to other sequences or to other sequences, such that the difference may be identified. Such conditions are sometimes called “stringent conditions.” Stringent conditions are sequence-dependent and can vary under different circumstances. For example, longer probe sequences generally hybridize to perfectly complementary sequences (over less than fully complementary sequences) at higher temperatures. Generally, stringent conditions may be selected to be about 5° C. lower than the thermal melting point (Tm) for the specific sequence at a defined ionic strength and pH. Exemplary stringent conditions may include those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Desired hybridization conditions may also be achieved with the addition of agents such as formamide or tetramethyl ammonium chloride (TMAC).

When using an array, one of skill in the art will appreciate that an enormous number of array designs are suitable for the practice of this invention. The array will typically include a number of test probes that specifically hybridize to the sequences of interest. That is, the array will include probes designed to hybridize to any region of the genes listed in Tables 1-8. In instances where the gene reference in the Tables is an EST, probes may be designed from that sequence or from other regions of the corresponding full-length transcript that may be available in any of the public sequence databases, such as those herein described. See WO 99/32660 for methods of producing probes for a given gene or genes. In addition, software is commercially available for designing specific probe sequences. Typically, the array will also include one or more control probes, such as probes specific for a constitutively expressed gene, thereby allowing data from different hybridizations to be normalized or controlled.

The hybridization-based assays may include, in addition to “test probes” (e.g., that bind the target sequences of interest, which are listed in Tables 1-8), the assay may also test for hybridization to one or a combination of control probes. Exemplary control probes include: normalization controls, expression level controls, and mismatch controls. For example, when determining the levels of gene expression in patient or control samples, the expression values may be normalized to control between samples. That is, the levels of gene expression in each sample may be normalized by determining the level of expression of at least one constitutively expressed gene in each sample. In accordance with the invention, the constitutively expressed gene is generally a transcript that is not differentially expressed in drug-sensitive versus drug-resistant samples.

Other useful controls are normalization controls, for example, using probes designed to be complementary to a labeled reference oligonucleotide added to the nucleic acid sample to be assayed. The signals obtained from the normalization controls after hybridization provide a control for variations in hybridization conditions, label intensity, “reading” efficiency and other factors that may cause the signal of a perfect hybridization to vary between arrays. In one embodiment, signals (e.g., fluorescence intensity) read from all other probes in the array are divided by the signal (e.g., fluorescence intensity) from the control probes thereby normalizing the measurements. Exemplary normalization probes are selected to reflect the average length of the other probes (e.g., test probes) present in the array, however, they may be selected to cover a range of lengths. The normalization control(s) may also be selected to reflect the (average) base composition of the other probes in the array. In some embodiments, the assay employs one or a few normalization probes, and they are selected such that they hybridize well (i.e., no secondary structure) and do not hybridize to any potential targets.

The hybridization-based assay may employ expression level controls, for example, probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

The hybridization-based assay may also employ mismatch controls for the target sequences, and/or for expression level controls or for normalization controls. Mismatch controls are probes designed to be identical to their corresponding test or control probes, except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20-mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present, the perfect match probes should provide a more intense signal than the mismatch probes. The difference in intensity between the perfect match and the mismatch probe helps to provide a good measure of the concentration of the hybridized material.

Alternatively, the invention may employ reverse transcription polymerase chain reaction (RT-PCR), which is a sensitive method for the detection of mRNA, including low abundant mRNAs present in clinical samples. The application of fluorescence techniques to RT-PCR combined with suitable instrumentation has led to quantitative RT-PCR methods that combine amplification, detection and quantification in a closed system. Two commonly used quantitative RT-PCR techniques are the Taqman RT-PCR assay (ABI, Foster City, USA) and the Lightcycler assay (Roche, USA).

Thus, in one embodiment of the present invention, the preparation of patient gene expression profiles or the preparation of drug-sensitive and drug-resistant profiles comprises conducting real-time quantitative PCR (TaqMan) with sample-derived RNA and control RNA. Holland, et al., PNAS 88:7276-7280 (1991) describe an assay known as a Taqman assay. The 5′ to 3′ exonuclease activity of Taq polymerase is employed in a polymerase chain reaction product detection system to generate a specific detectable signal concomitantly with amplification. An oligonucleotide probe, non-extendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. A version of this assay is also described in Gelfand et al., in U.S. Pat. No. 5,210,015, which is hereby incorporated by reference.

Further, U.S. Pat. No. 5,491,063 to Fisher, et al., which is hereby incorporated by reference, provides a Taqman-type assay. The method of Fisher et al. provides a reaction that results in the cleavage of single-stranded oligonucleotide probes labeled with a light-emitting label wherein the reaction is carried out in the presence of a DNA binding compound that interacts with the label to modify the light emission of the label. The method of Fisher uses the change in light emission of the labeled probe that results from degradation of the probe.

The TaqMan detection assays offer certain advantages. First, the methodology makes possible the handling of large numbers of samples efficiently and without cross-contamination and is therefore adaptable for robotic sampling. As a result, large numbers of test samples can be processed in a very short period of time using the TaqMan assay. Another advantage of the TaqMan system is the potential for multiplexing. Since different fluorescent reporter dyes can be used to construct probes, the expression of several different genes associated with drug sensitivity or resistance may be assayed in the same PCR reaction, thereby reducing the labor costs that would be incurred if each of the tests were performed individually. Thus, the TaqMan assay format is preferred where the patient's gene expression profile, and the corresponding drug-sensitive and drug-resistance profiles comprise the expression levels of about 20 of fewer, or about 10 or fewer, or about 7 of fewer, or about 5 genes (e.g., genes listed in one or more of Tables 1-8).

Alternatively, the assay format may employ the methodologies described in Direct Multiplexed Measurement of Gene Expression with Color-Coded Probe Pairs, Nature Biotechnology (Mar. 7, 2008), which describes the nCounter™ Analysis System (nanoString Technologies). This system captures and counts individual mRNA transcripts by a molecular bar-coding technology, and is commercialized by Nanostring.

In other embodiments, the invention employs detection and quantification of RNA levels in real-time using nucleic acid sequence based amplification (NASBA) combined with molecular beacon detection molecules. NASBA is described for example, in Compton J., Nucleic acid sequence-based amplification, Nature 1991;350(6313):91-2. NASBA is a singe-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In yet other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct mRNA capture with branched DNA (QuantiGene™, Panomics) or Hybrid Capture™ (Digene).

The design of appropriate probes for hybridizing to a particular target nucleic acid, and as configured for any appropriate nucleic acid detection assay, is well known.

### Computer System

In another aspect, the invention is a computer system that contains a database, on a computer-readable medium, of gene expression values indicative of a tumor's drug-resistance and/or drug-sensitivity. These gene expression values are determined (as already described) in established cell lines, cell cultures established from patient samples, or directly from patient specimens, and for genes selected from one or more of Tables 1-8. The database may include, for each gene, sensitive and resistant gene expression levels, thresholds, or Mean values, as well as various statistical measures, including measures of value dispersion (e.g., Standard Variation), fold change (e.g., between sensitive and resistant samples), and statistical significance (statistical association with drug sensitivity or resistance). Generally, signatures may be assembled based upon parameters to be selected and input by a user, with these parameters including of cancer or tumor type, histology, and/or candidate chemotherapeutic agents or combinations.

In certain embodiments, the database contains mean gene expression values for at least about 5, 7, 10, 20, 40, 50, or 100 genes selected from any one, or a combination of, Tables 1-8. In some embodiments, the database may contain mean gene expression values for more than about 100 genes, or about 300 genes, or about 400 genes selected from Tables 1-8. In one embodiment, the database contains mean gene expression values for all or substantially all the genes listed in Tables 1-8.

The computer system of the invention may be programmed to compare, score, or classify (e.g., in response to user inputs) a gene expression profile against a drug-sensitive gene expression signature and/or a drug-resistant gene expression signature stored and/or generated from the database, to determine whether the gene expression profile is itself a drug sensitive or drug-resistant profile. For example, the computer system may be programmed to perform any of the known classification schemes for classifying gene expression profiles. Various classification schemes are known for classifying samples, and these include, without limitation: Principal Components Analysis, Naïve Bayes, Support Vector Machines, Nearest Neighbors, Decision Trees, Logistic, Artificial Neural Networks, and Rule-based schemes. The computer system may employ a classification algorithm or “class predictor” as described in R. Simon, Diagnostic and prognostic prediction using gene expression profiles in high-dimensional microarray data, British Journal of Cancer (2003) 89, 1599-1604, which is hereby incorporated by reference in its entirety.

The computer system of the invention may comprise a user interface, allowing a user to input gene expression values for comparison to a drug-sensitive and/or drug-resistant gene expression profile. The patient's gene expression values may be input from a location remote from the database.

The computer system may further comprise a display, for presenting and/or displaying a result, such as a signature assembled from the database, or the result of a comparison (or classification) between input gene expression values and a drug-sensitive and drug-resistant signatures. Such results may further be provided in any form (e.g., as a printable or printed report).

The computer system of the invention may further comprise relational databases containing sequence information, for instance, for the genes of Tables 1-8. For example, the database may contain information associated with a given gene, cell line, or patient sample used for preparing gene signatures, such as descriptive information about the gene associated with the sequence information, or descriptive information concerning the clinical status of the patient (e.g., treatment regimen and outcome). The database may be designed to include different parts, for instance a sequence database and a gene expression database. Methods for the configuration and construction of such databases and computer-readable media to which such databases are saved are widely available, for instance, see U.S. Pat. No. 5,953,727, which is hereby incorporated by reference in its entirety.

The databases of the invention may be linked to an outside or external database (e.g., on the world wide web) such as GenBank (ncbi.nlm.nih.gov/entrez.index.html); KEGG (genome.ad.jp/kegg); SPAD (grt.kuyshu-u.ac.jp/spad/index.html); HUGO (gene.ucl.ac.uk/hugo); Swiss-Prot (expasy.ch.sprot); Prosite (expasy.ch/tools/scnpsitl.html); OMIM (ncbi.nlm.nih.gov/omim); and GDB (gdb.org). In certain embodiments, the external database is GenBank and the associated databases maintained by the National Center for Biotechnology Information (NCBI) (ncbi.nlm.nih.gov).

Any appropriate computer platform, user interface, etc. may be used to perform the necessary comparisons between sequence information, gene expression information (e.g., gene expression profiles) and any other information in the database or information provided as an input. For example, a large number of computer workstations are available from a variety of manufacturers, such has those available from Silicon Graphics. Client/server environments, database servers and networks are also widely available and appropriate platforms for the databases described herein.

The databases of the invention may be used to produce, among other things, electronic Northerns that allow the user to determine the samples in which a given gene is expressed and to allow determination of the abundance or expression level of the given gene.

### Diagnostic Kits

The invention further provides a kit or probe array containing nucleic acid primers and/or probes for determining the level of expression in a patient tumor specimen or cell culture of a plurality of genes listed in Tables 1-8. The probe array may contain 3000 probes or less, 2000 probes or less, 1000 probes or less, or 500 probes or less, so to embody a custom set for preparing gene expression profiles as described herein. In some embodiments, the kit may consist essentially of primers and/or probes related to evaluating drug-sensitivity/resistant in a sample, and primers and/or probes related to necessary or meaningful assay controls (such as expression level controls and normalization controls, as described herein under “Gene Expression Assay Formats”). The kit for evaluating drug-sensitivity/resistance may comprise nucleic acid probes and/or primers designed to detect the expression level of ten or more genes associated with drug sensitivity/resistance, such as the genes listed in Tables 1-8. The kit may include a set of probes and/or primers designed to detect or quantify the expression levels of at least 5, 7, 10, 20, 100, 200, 250, or 400 genes listed in one or more of Tables 1-8. The primers and/or probes may be designed to detect gene expression levels in accordance with any assay format, including those described herein under the heading “Assay Format.” Exemplary assay formats include polymerase-based assays, such as RT-PCR, Taqman™, hybridization-based assays, for example using DNA microarrays or other solid support, nucleic acid sequence based amplification (NASBA), flap endonuclease-based assays. The kit need not employ a DNA microarray or other high density detection format.

In accordance with this aspect, the probes and primers may comprise antisense nucleic acids or oligonucleotides that are wholly or partially complementary to the diagnostic targets described herein (e.g., Tables 1-8). The probes and primers will be designed to detect the particular diagnostic target via an available nucleic acid detection assay format, which are well known in the art. The kits of the invention may comprise probes and/or primers designed to detect the diagnostic targets via detection methods that include amplification, endonuclease cleavage, and hybridization.

### Examples

### Example 1

### Identifying Gene Expression Signatures

Cancer cell lines (breast cancer) from a Berkeley Labs collection (Neve et al., A collection of breast cancer cell lines for the study of functionally distinct cancer subtypes. Cancer Cell, 10, 6:515-27 (2006)) were tested for their sensitivity in vitro to the combinations TFAC, EC, AC, and ACT. TFAC is the combination of cyclophosphamide, doxorubicin, fluorouracil, and paclitaxel. EC is the combination of cyclophosphamide and epirubicin. AC is the combination of cyclophosphamide and doxorubicin. ACT is the combination of cyclophosphamide, docetaxel, and doxorubicin. In vitro chemosensitivity was determined using the ChemoFx™ assay (Precision Therapeutics, Inc., Pittsburgh, Pa.).

| Cell Line Name | ATCC Deposit Number |
|---|---|
| AU565 | CRL-2351 |
| BT20 | HTB-19 |
| BT474 | HTB-20 |
| BT483 | HTB-121 |
| BT549 | HTB-122 |
| CAMA1 | HTB-21 |
| HCC1143 | CRL-2321 |
| HCC1187 | CRL-2322 |
| HCC1428 | CRL-2327 |
| HCC1500 | CRL-2329 |
| HCC1569 | CRL-2330 |
| HCC1937 | CRL-2336 |
| HCC1954 | CRL-2338 |
| HCC202 | CRL-2316 |
| HCC38 | CRL-2314 |
| MCF10A | CRL-10317 |
| MCF7 | HTB-22 |
| MDAMB157 | HTB-24 |
| MDAMB175VII | HTB-25 |
| MDAMB231 | HTB-26 |
| MDAMB361 | HTB-27 |
| MDAMB415 | HTB-128 |
| MDAMB436 | HTB-130 |
| MDAMB453 | HTB-131 |
| MDAMB468 | HTB-132 |
| SKBR3 | HTB-30 |
| T47D | HTB-133 |
| UACC812 | CRL-1897 |
| ZR751 | CRL-1500 |

The results of the sensitivity testing of cell lines against TFAC are shown below, designating the top ⅓ lines as sensitive and bottom ⅓ lines as resistant (smaller aAUC corresponds to higher sensitivity to drug):

| Cell Line | aAUC Score for TFAC |
|---|---|
| SKBR3 | 4.095892 |
| MDAMB231 | 4.377128 |
| HCC202 | 4.613757 |
| MDAMB468 | 4.625136 |
| AU565 | 4.683571 |
| HCC38 | 4.702548 |
| T47D | 4.747912 |
| HCC1954 | 5.338308 |
| MDAMB157 | 5.426692 |
| BT549 | 5.547992 |
| MCF10A | 5.659951 |
| HCC1937 | 6.038998 |
| HCC1143 | 6.310999 |
| UACC812 | 6.4998 |
| HCC1187 | 6.701829 |
| BT474 | 6.84609 |
| MCF7 | 6.849411 |
| MDAMB436 | 6.88757 |
| HCC1569 | 7.12799 |
| BT20 | 7.21069 |
| ZR751 | 7.552486 |
| CAMA1 | 7.814893 |
| MDAMB453 | 8.354905 |
| HCC1428 | 8.792317 |
| HCC1500 | 8.899185 |
| MDAMB415 | 8.926433 |
| MDAMB175VII | 9.00102 |
| MDAMB361 | 9.370602 |
| BT483 | 10.627008 |

The results of the sensitivity testing of cell lines against EC are shown below, designating the top ⅓ lines as sensitive and bottom ⅓ lines as resistant (smaller aAUC corresponds to higher sensitivity to drug):

| Cell line | aAUC Score for EC |
|---|---|
| SKBR3 | 3.791795 |
| MDAMB231 | 3.977075 |
| MDAMB468 | 4.369188 |
| MDAMB157 | 4.395603 |
| T47D | 4.69654 |
| HCC1954 | 4.710457 |
| HCC38 | 4.714469 |
| BT549 | 5.056839 |
| AU565 | 5.20695 |
| HCC1187 | 5.448001 |
| HCC202 | 5.591795 |
| MCF10A | 5.60165 |
| HCC1937 | 6.035871 |
| HCC1143 | 6.057825 |
| MDAMB436 | 6.38585 |
| UACC812 | 6.494965 |
| MCF7 | 6.518678 |
| CAMA1 | 6.525865 |
| BT20 | 6.908749 |
| HCC1569 | 6.928442 |
| ZR751 | 7.140626 |
| BT474 | 7.854797 |
| HCC1428 | 8.411347 |
| MDAMB175VII | 8.438685 |
| MDAMB415 | 8.530483 |
| HCC1500 | 8.843713 |
| MDAMB361 | 9.097793 |
| MDAMB453 | 9.169443 |
| BT483 | 9.802551 |

The results of the sensitivity testing of cell lines against AC are shown below, designating the top ⅓ lines as sensitive and bottom ⅓ lines as resistant (smaller aAUC corresponds to higher sensitivity to drug):

| Cell Lines | aAUC Score for AC |
|---|---|
| MDAMB231 | 4.156278 |
| SKBR3 | 4.50647 |
| MDAMB468 | 4.722416 |
| MDAMB157 | 4.937313 |
| AU565 | 5.102452 |
| T47D | 5.142585 |
| HCC1954 | 5.798878 |
| HCC202 | 5.875344 |
| HCC1187 | 5.908905 |
| HCC38 | 6.016731 |
| BT549 | 6.385796 |
| MCF10A | 6.728466 |
| HCC1143 | 6.760749 |
| HCC1937 | 7.040271 |
| MDAMB436 | 7.054871 |
| MCF7 | 7.378485 |
| UACC812 | 7.550414 |
| BT474 | 7.739023 |
| HCC1569 | 7.740798 |
| ZR751 | 8.020471 |
| BT20 | 8.101519 |
| CAMA1 | 8.416588 |
| HCC1428 | 9.06864 |
| MDAMB361 | 9.521091 |
| MDAMB415 | 9.569508 |
| HCC1500 | 10.03434 |
| MDAMB175VII | 10.74181 |
| BT483 | 10.96079 |
| MDAMB453 | 11.48155 |

The results of the sensitivity testing of cell lines against ACT are shown below, designating the top ⅓ lines as sensitive and bottom ⅓ lines as resistant (smaller aAUC corresponds to higher sensitivity to drug):

| Cell Line | aAUC Score for ACT |
|---|---|
| SKBR3 | 3.871316 |
| MDAMB468 | 4.282198 |
| MDAMB231 | 5.191808 |
| HCC38 | 5.237426 |
| UACC812 | 5.378436 |
| HCC1954 | 5.545022 |
| MCF10A | 5.690861 |
| AU565 | 5.815504 |
| T47D | 5.83053 |
| BT549 | 6.066557 |
| MDAMB436 | 6.170432 |
| MDAMB415 | 6.250316 |
| MCF7 | 6.592258 |
| HCC1143 | 6.740768 |
| BT20 | 6.862321 |
| HCC1569 | 6.988493 |
| BT483 | 7.07682 |
| CAMA1 | 7.44813 |
| HCC1937 | 8.055724 |
| BT474 | 8.517679 |
| HCC1428 | 8.518428 |
| ZR751 | 8.643591 |
| MDAMB453 | 8.847987 |
| MDAMB175VII | 9.086667 |
| HCC1500 | 9.613739 |
| MDAMB361 | 9.881265 |
| HCC1187 | NaN |
| HCC202 | NaN |
| MDAMB157 | NaN |

The aAUC scores for all cell lines across the four drug combinations were as follows, illustrating the level of variance between combinations and cell lines:

| Cell Line | TFAC | EC | AC | ACT |
|---|---|---|---|---|
| AU565 | 4.683571 | 5.20695 | 5.102452 | 5.815504 |
| BT20 | 7.21069 | 6.908749 | 8.101519 | 6.862321 |
| BT474 | 6.84609 | 7.854797 | 7.739023 | 8.517679 |
| BT483 | 10.627008 | 9.802551 | 10.960787 | 7.07682 |
| BT549 | 5.547992 | 5.056839 | 6.385796 | 6.066557 |
| CAMA1 | 7.814893 | 6.525865 | 8.416588 | 7.44813 |
| HCC1143 | 6.310999 | 6.057825 | 6.760749 | 6.740768 |
| HCC1187 | 6.701829 | 5.448001 | 5.908905 | NaN |
| HCC1428 | 8.792317 | 8.411347 | 9.06864 | 8.518428 |
| HCC1500 | 8.899185 | 8.843713 | 10.034344 | 9.613739 |
| HCC1569 | 7.12799 | 6.928442 | 7.740798 | 6.988493 |
| HCC1937 | 6.038998 | 6.035871 | 7.040271 | 8.055724 |
| HCC1954 | 5.338308 | 4.710457 | 5.798878 | 5.545022 |
| HCC202 | 4.613757 | 5.591795 | 5.875344 | NaN |
| HCC38 | 4.702548 | 4.714469 | 6.016731 | 5.237426 |
| MCF10A | 5.659951 | 5.60165 | 6.728466 | 5.690861 |
| MCF7 | 6.849411 | 6.518678 | 7.378485 | 6.592258 |
| MDAMB157 | 5.426692 | 4.395603 | 4.937313 | NaN |
| MDAMB175VII | 9.00102 | 8.438685 | 10.741814 | 9.086667 |
| MDAMB231 | 4.377128 | 3.977075 | 4.156278 | 5.191808 |
| MDAMB361 | 9.370602 | 9.097793 | 9.521091 | 9.881265 |
| MDAMB415 | 8.926433 | 8.530483 | 9.569508 | 6.250316 |
| MDAMB436 | 6.88757 | 6.38585 | 7.054871 | 6.170432 |
| MDAMB453 | 8.354905 | 9.169443 | 11.48155 | 8.847987 |
| MDAMB468 | 4.625136 | 4.369188 | 4.722416 | 4.282198 |
| SKBR3 | 4.095892 | 3.791795 | 4.50647 | 3.871316 |
| T47D | 4.747912 | 4.69654 | 5.142585 | 5.83053 |
| UACC812 | 6.4998 | 6.494965 | 7.550414 | 5.378436 |
| ZR751 | 7.552486 | 7.140626 | 8.020471 | 8.643591 |

These in vitro chemosensitivity results were used to determine gene signatures indicative of sensitivity and resistance to the selected combinations, using publicly available gene expression data. The gene expression data had been determined using the hgu133a microarray format (Affymetrix). The raw gene expression values are shown in Tables 1, 3, 5, and 7, respectively for each drug combination. Gene signatures were identified by Principal Components Analysis, with cut-offs determined by classifier algorithm.

Tables 2, 4, 6, and 8 each provide the mean gene expression values for sensitive cell lines, and the mean gene expression values for resistant cell lines, for each combination of therapeutic agents. The Tables also provide the fold change from sensitive to resistant. For example, where x is the mean expression score for sensitive cell lines for a particular gene, and y is the mean expression score for resistant cell lines for that gene, fold change is represented by mean X/mean Y.

Tables 9-12 each provide an Entrez ID and weight score for each gene of the signatures disclosed in Tables 1 and 2, 3 and 4, 5 and 6, and 7 and 8, respectively.

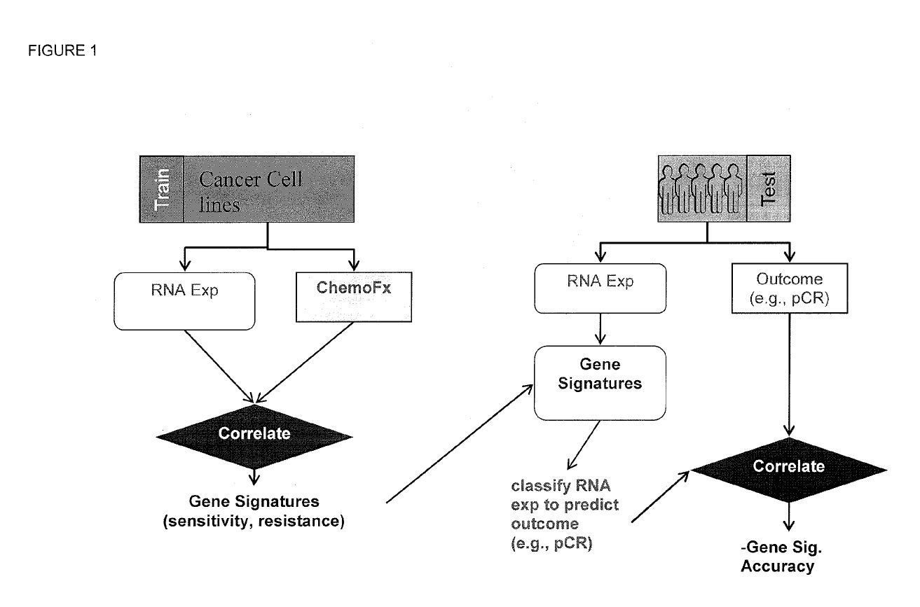

The procedure for identifying gene expression signatures is shown diagrammatically in FIG. 1.

### Example 2

### Validating Gene Expression Signatures

The gene expression signatures resulting from the above analysis were validated in patient populations by comparing publicly available patient tumor gene expression data (based on hgu133a microarray platform) with the corresponding outcome of treatment with TFAC, EC, AC, and ACT. The validation sets were as follows.

133 neoadjuvant breast cancer patients, treated with TFAC, and outcomes evaluated for pCR (“Pusztai set”). Hess, K R, Anderson, K, Symmans, W F, Valero, V, Ibrahim, N, Mejia, J A, Booser, D, Theriault, R L, Buzdar, A U, Dempsey, P J, Rouzier, R, Sneige, N, Ross, J S, Vidaurre, T, Gomez, H L, Hortobagyi, G N, Pusztai, L (2006). Pharmacogenomic predictor of sensitivity to preoperative chemotherapy with paclitaxel and fluorouracil, doxorubicin, and cyclophosphamide in breast cancer. J. Clin. Oncol., 24, 26:4236-44.

37 neoadjuvant breast cancer patients, treated with EC, and outcomes evaluated for pCR (“Bertheau set”). Bertheau, P, Turpin, E, Rickman, D S, Espié, M, de Reyniès, A, Feugeas, J P, Plassa, L F, Soliman, H, Varna, M, de Roquancourt, A, Lehmann-Che, J, Beuzard, Y, Marty, M, Misset, J L, Janin, A, de Thé, H (2007). Exquisite sensitivity of TP53 mutant and basal breast cancers to a dose-dense epirubicin-cyclophosphamide regimen. PLoS Med., 4, 3:e90.

326 neoadjuvant breast cancer patients treated with AC or ACT, and outcomes evaluated for pCR (“Paik set”). Bear, H D, Anderson, S, Brown, A, Smith, R, Mamounas, E P, Fisher, B, Margolese, R, Theoret, H, Soran, A, Wickerham, D L, Wolmark, N (2003). The effect on tumor response of adding sequential preoperative docetaxel to preoperative doxorubicin and cyclophosphamide: preliminary results from National Surgical Adjuvant Breast and Bowel Project Protocol B-27. J. Clin. Oncol., 21, 22:4165-74.

The data sets for validation are summarized as follows:

| | | | | no. | no. |
|---|---|---|---|---|---|
| | | | Out- | patients | patients |
| | Platform | Drug | come | pCR | non-pCR |
| Pusztai | Hgu133a | TFAC | pCR | 34 (19%) | 98 |
| Bertheau | Hgu133a | EC | pCR | 9 (25.7%) | 26 |
| Paik (AC) | Hgu133a + 2 | AC | pCR | 22 (10%) | 199 |
| Paik (ACT) | Hgu133a + 2 | ACT | pCR | 25 (24%) | 78 |

Patient samples were classified as resistant and/or sensitive to the chemotherapeutic agent combinations by scoring the publicly available gene expression data against the identified gene signatures, thereby obtaining an outcome prediction. Bair, E, Tibshirani, R (2004). Semi-supervised methods to predict patient survival from gene expression data. PLoS Biol., 2, 4:E108. Specifically, standard regression coefficients for each gene in the training set were calculated; genes were selected having a coefficient larger than the threshold, where the threshold is estimated by cross-validation in the training set; a reduced data matrix on these selected genes was formed; the first principal components based on the reduced data matrix was calculated; and the first principal component was used in a regression model to predict the patient's outcome. The accuracy of the classification or prediction was validated by comparing the prediction with the actual outcome of treatment.

The accuracy of the gene signatures were as follows.

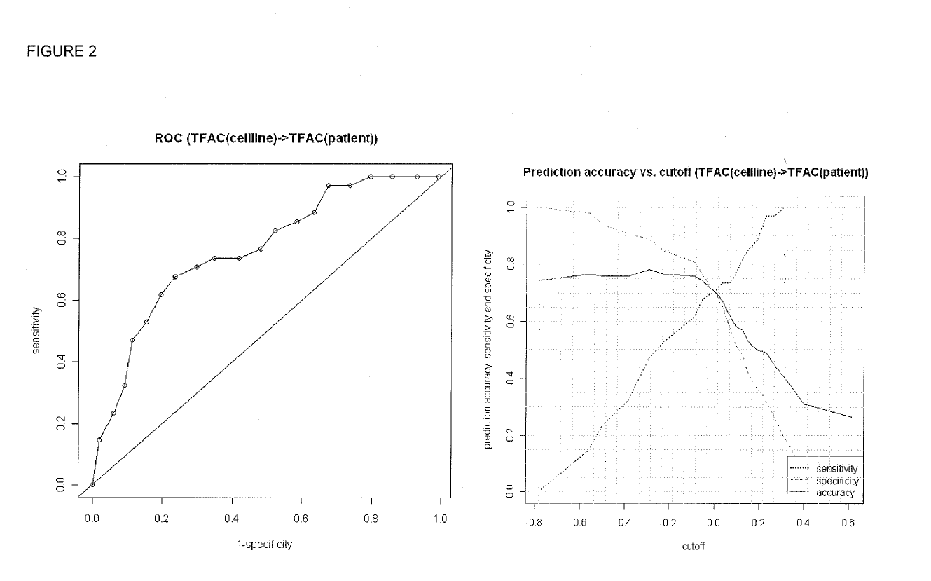

The accuracy of a 423-gene signature from Tables 1 and 2 for predicting pCR in the Pusztai data set was determined, and is shown in FIG. 2. The results are shown as a receiver operator curve (ROC).

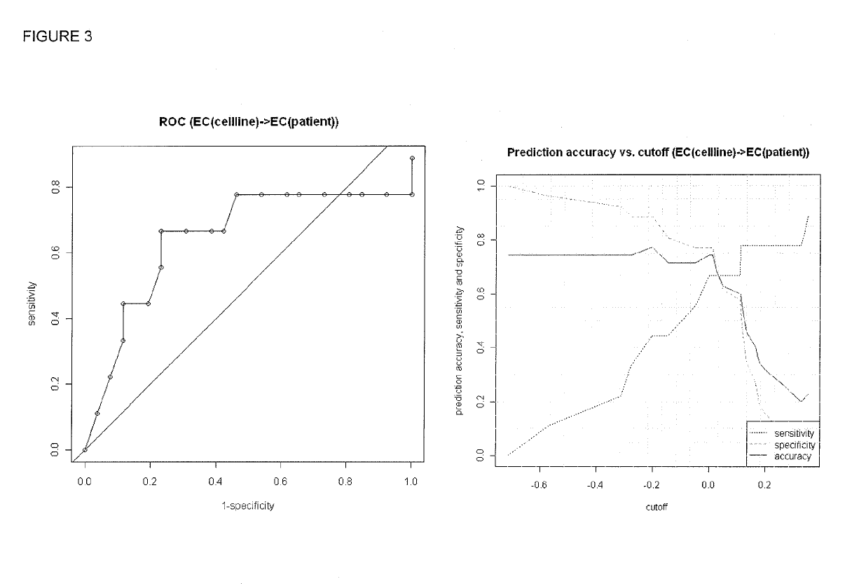

The accuracy of a 370-gene signature from Tables 3 and 4 for predicting pCR in the Bertheau data set was determined, and is shown in FIG. 3. The results are shown as a receiver operator curve (ROC).

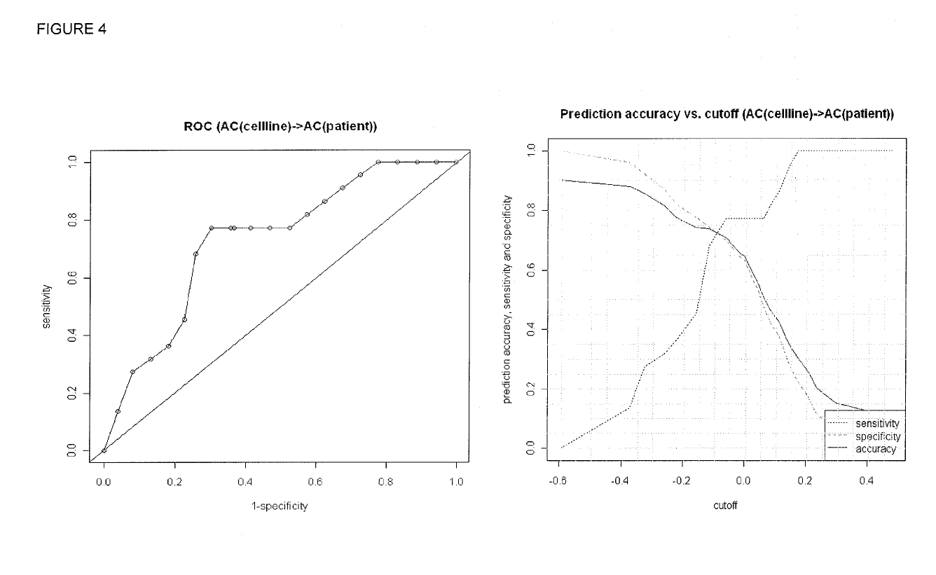

The accuracy of a 371-gene signature from Tables 5 and 6 for predicting pCR in the Paik data set (AC) was determined, and is shown in FIG. 4. The results are shown as a receiver operator curve (ROC).

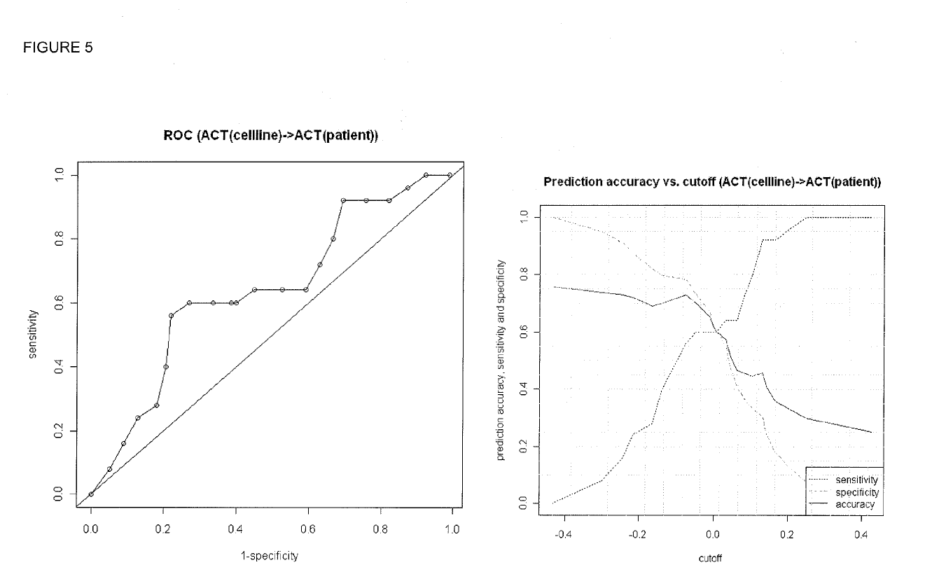

The accuracy of a 402-gene signature from Tables 5 and 6 for predicting pCR in the Paik data set (ACT) was determined, and is shown in FIG. 5. The results are shown as a receiver operator curve (ROC).

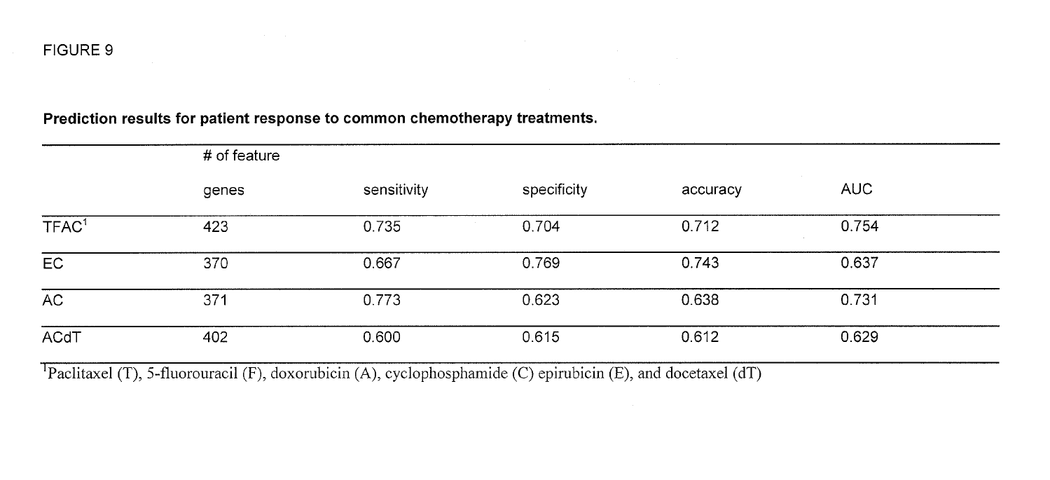

The accuracy of each multigene predictor is summarized in FIG. 9.

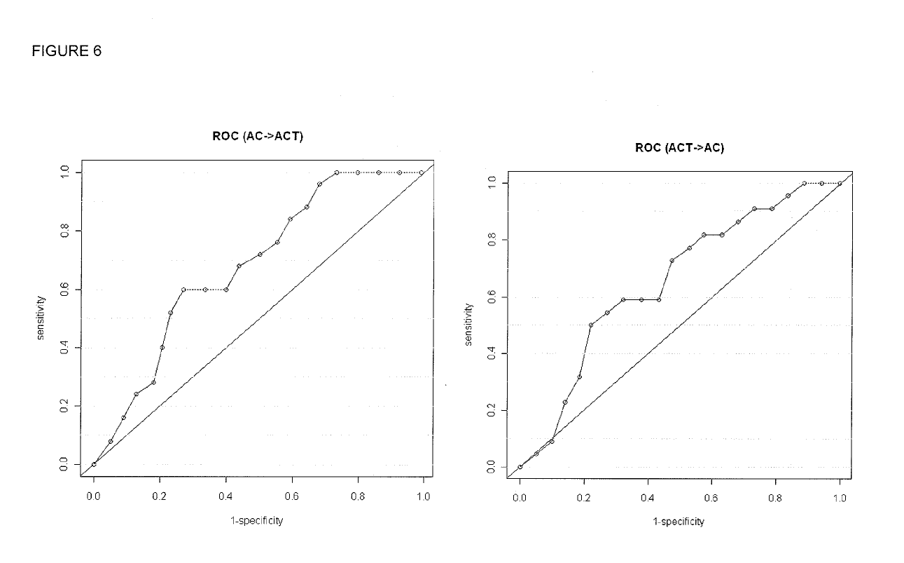

FIG. 6 illustrates the accuracy of the AC-trained signature of Tables 5 and 6 for predicting pCR upon treatment with ACT (left panel), and the accuracy of the ACT-trained signature of Tables 6 and 7 for predicting pCR upon treatment with AC (right panel). Results are shown as ROCs, and illustrate that the gene signature has a predictive element, in addition to prognostic.

Signatures were also trained with a variety of chemotherapeutic agents, and then tested for predictive accuracy against the AC, ACT, EC, and TFAC patient data sets. The predictions are summarized in the following Table, showing that the gene expression signatures have a predictive element:

| Drug.training/drug.test | TFAC | EC | AC | ACT |
|---|---|---|---|---|
| Cis/Cyclo | 0.659091 | 0.485714 | 0.5384615 | 0.6019417 |
| Cycle | 0.689394 | 0.714286 | 0.638009 | 0.6019417 |
| ACT | 0.651515 | 0.542857 | 0.561086 | 0.6116505 |
| AC | 0.689394 | 0.742857 | 0.6470588 | 0.6504854 |
| TFAC | 0.704546 | 0.714286 | 0.5972851 | 0.6407767 |
| EC | 0.674242 | 0.742857 | 0.6289593 | 0.6601942 |
| FEC | 0.704546 | 0.685714 | 0.6063348 | 0.6504854 |
| TFEC | 0.636364 | 0.542857 | 0.5565611 | 0.6213592 |
| docetaxel | 0.643939 | 0.514286 | 0.60181 | 0.5825243 |
| doxorubicin | 0.628788 | 0.685714 | 0.6063348 | 0.6019417 |
| epirubicin | 0.666667 | 0.657143 | 0.638009 | 0.6019417 |
| epirubicin/paclitaxel | 0.621212 | 0.628571 | 0.6199095 | 0.631068 |
| 5-FU | 0.636364 | 0.457143 | 0.5656109 | 0.5728155 |
| Gemcitabine | 0.643939 | 0.542857 | 0.5746606 | 0.5339806 |
| Irinotecan | 0.621212 | 0.428571 | 0.520362 | 0.5631068 |
| TAXOL | 0.659091 | 0.542857 | 0.6289593 | 0.6019417 |

### Example 3

### Testing the Stability of the Multigene Predictors

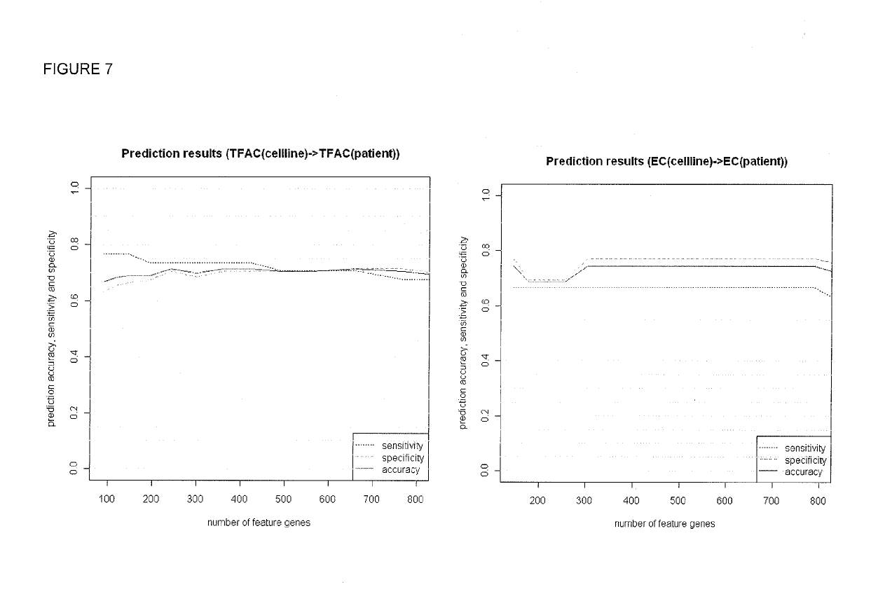

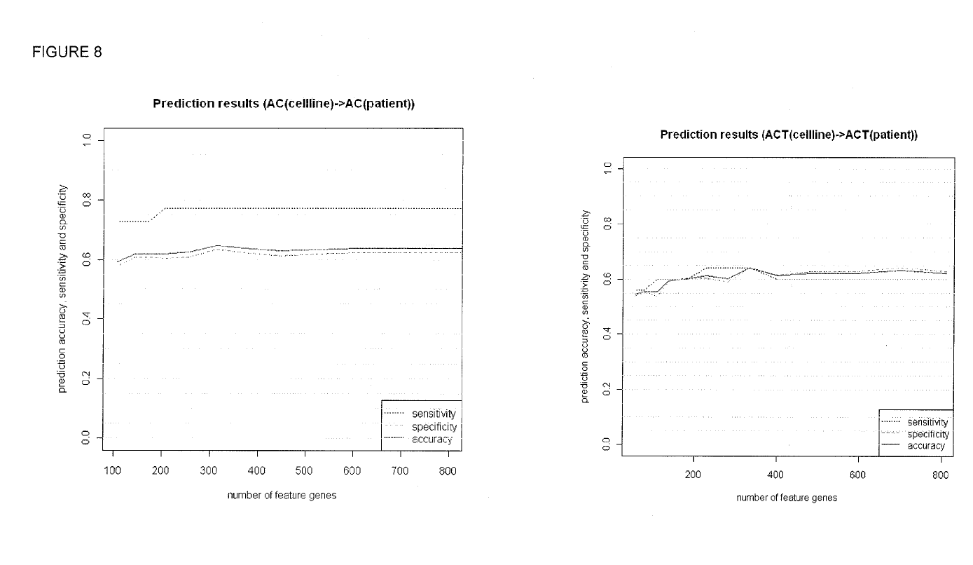

The stability of the multigene predictors were tested with a sensitivity analysis, and the results shown in FIGS. 7 and 8.

The left panel of FIG. 7 shows that the gene expression signature of Tables 1 and 2 is stable over a large range of increasing gene number, from less than about 10 to over 400 genes. The right panel shows that the gene expression signature of Tables 3 and 4 is stable over a large range of gene number, from about 100 to over about 400 genes.

The left panel of FIG. 8 shows that the gene expression signature of Tables 5 and 6 are stable over a large range of increasing gene number, from less than about 10 to over 400 genes. The right panel shows that the gene expression signature of Tables 7 and 8 are stable over a large range of gene number, from about 10 up to about 400 genes.

Without further description, it is believed that one of ordinary skill in the art can, using the preceding description and illustrative examples, practice the invention including as claimed below.

All references cited herein are hereby incorporated by reference in their entireties and for all purposes.

Lengthy table referenced here
US20100331210A1-20101230-T00001
Please refer to the end of the specification for access instructions.

Lengthy table referenced here
US20100331210A1-20101230-T00002
Please refer to the end of the specification for access instructions.

Lengthy table referenced here
US20100331210A1-20101230-T00003
Please refer to the end of the specification for access instructions.

Lengthy table referenced here
US20100331210A1-20101230-T00004
Please refer to the end of the specification for access instructions.

Lengthy table referenced here
US20100331210A1-20101230-T00005
Please refer to the end of the specification for access instructions.

Lengthy table referenced here
US20100331210A1-20101230-T00006
Please refer to the end of the specification for access instructions.

Lengthy table referenced here
US20100331210A1-20101230-T00007
Please refer to the end of the specification for access instructions.

Lengthy table referenced here
US20100331210A1-20101230-T00008
Please refer to the end of the specification for access instructions.

Lengthy table referenced here
US20100331210A1-20101230-T00009
Please refer to the end of the specification for access instructions.

Lengthy table referenced here
US20100331210A1-20101230-T00010
Please refer to the end of the specification for access instructions.

Lengthy table referenced here
US20100331210A1-20101230-T00011
Please refer to the end of the specification for access instructions.

Lengthy table referenced here
US20100331210A1-20101230-T00012
Please refer to the end of the specification for access instructions.

Lengthy table referenced here
US20100331210A1-20101230-T00013
Please refer to the end of the specification for access instructions.

Lengthy table referenced here
US20100331210A1-20101230-T00014
Please refer to the end of the specification for access instructions.

Lengthy table referenced here
US20100331210A1-20101230-T00015
Please refer to the end of the specification for access instructions.

Lengthy table referenced here
US20100331210A1-20101230-T00016
Please refer to the end of the specification for access instructions.

Lengthy table referenced here
US20100331210A1-20101230-T00017
Please refer to the end of the specification for access instructions.

Lengthy table referenced here
US20100331210A1-20101230-T00018
Please refer to the end of the specification for access instructions.

Lengthy table referenced here
US20100331210A1-20101230-T00019
Please refer to the end of the specification for access instructions.

Lengthy table referenced here
US20100331210A1-20101230-T00020
Please refer to the end of the specification for access instructions.

Lengthy table referenced here
US20100331210A1-20101230-T00021
Please refer to the end of the specification for access instructions.

Lengthy table referenced here
US20100331210A1-20101230-T00022
Please refer to the end of the specification for access instructions.

Lengthy table referenced here
US20100331210A1-20101230-T00023
Please refer to the end of the specification for access instructions.

Lengthy table referenced here
US20100331210A1-20101230-T00024
Please refer to the end of the specification for access instructions.

Lengthy table referenced here
US20100331210A1-20101230-T00025
Please refer to the end of the specification for access instructions.

Lengthy table referenced here
US20100331210A1-20101230-T00026
Please refer to the end of the specification for access instructions.

Lengthy table referenced here
US20100331210A1-20101230-T00027
Please refer to the end of the specification for access instructions.

Lengthy table referenced here
US20100331210A1-20101230-T00028
Please refer to the end of the specification for access instructions.

Lengthy table referenced here
US20100331210A1-20101230-T00029
Please refer to the end of the specification for access instructions.

Lengthy table referenced here
US20100331210A1-20101230-T00030
Please refer to the end of the specification for access instructions.

Lengthy table referenced here
US20100331210A1-20101230-T00031
Please refer to the end of the specification for access instructions.

Lengthy table referenced here
US20100331210A1-20101230-T00032
Please refer to the end of the specification for access instructions.

Lengthy table referenced here
US20100331210A1-20101230-T00033
Please refer to the end of the specification for access instructions.

Lengthy table referenced here
US20100331210A1-20101230-T00034
Please refer to the end of the specification for access instructions.

Lengthy table referenced here
US20100331210A1-20101230-T00035
Please refer to the end of the specification for access instructions.

Lengthy table referenced here
US20100331210A1-20101230-T00036
Please refer to the end of the specification for access instructions.

Lengthy table referenced here
US20100331210A1-20101230-T00037
Please refer to the end of the specification for access instructions.

Lengthy table referenced here
US20100331210A1-20101230-T00038
Please refer to the end of the specification for access instructions.

Lengthy table referenced here
US20100331210A1-20101230-T00039
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20100331210A1). An electronic copy of the table will also be available from the USPTO upon request and payment of the fee set forth in 37 CFR 1.19(b)(3).

1. A method for preparing a gene expression profile indicative of drug-sensitivity or drug-resistance, comprising:
extracting RNA from a patient tumor specimen or cells cultured therefrom, and
determining the level of expression for at least 5 genes listed in one or more of Tables 1-8, thereby preparing the gene expression profile.

2. The method of claim 1, wherein the tumor is derived from a tissue selected from breast, ovaries, lung, colon, skin, prostate, kidney, endometrium, nasopharynx, pancreas, head and neck, kidney, and brain.

3. The method of claim 1, wherein the tumor specimen is a carcinoma.

4. The method of any one of claims 1 to 3, wherein the specimen is obtained by surgery or biopsy, or is obtained from blood or ascites.

5. The method of claim 4, wherein the tumor specimen is a breast tumor specimen.

6. The method of any one of claims 1 to 5, wherein the patient has primary cancer.

7. The method of any one of claims 1 to 5, wherein the patient has recurrent cancer.

8. The method of claim 1, wherein the patient is a candidate for treatment with a combination selected from: cyclophosphamide, doxorubicin, fluorouracil, and paclitaxel (TFAC); cyclophosphamide and epirubicin (EC); cyclophosphamide and doxorubicin (AC); cyclophosphamide, docetaxel, and doxorubicin (ACT).

9. The method of any one of claims 1 to 8, wherein the RNA is extracted from a tumor specimen.

10. The method of claim 9, wherein the tumor specimen is formalin-fixed and paraffin-embedded.

11. The method of any one of claims 1 to 8, wherein the RNA is extracted from cultured cells derived from the tumor specimen.

12. The method of claim 11, wherein the cultured cells are enriched for malignant cells.

13. The method of claim 12, wherein the cultured cells are grown in a monolayer culture from a plurality of explants of the tumor specimen.

14. The method of any one of claims 1 to 13, wherein the levels of expression are determined by hybridizing nucleic acids to oligonucleotide probes, by RT-PCR, or by direct mRNA capture.

15. The method of any one of claims 1 to 14, wherein the RNA is total RNA.

16. The method of any one of claims 1 to 14, wherein the RNA is polyA+RNA.

17. The method of any one of claims 1 to 16, wherein the RNA is reverse transcribed are/or amplified.

18. The method of any one of claims 1 to 17, wherein the gene expression profile comprises the level of expression for at least about 10 genes listed in one or more of Tables 1-8.

19. The method of claim 18, wherein the gene expression profile comprises the level of expression for at least about 100 genes listed in one or more of Tables 1-8.

20. The method of claim 18, wherein the gene expression profile comprises the level of expression for at least about 200 genes listed in one or more of Tables 1-8.

21. The method of claim 18, wherein the at least 10 genes are listed in Tables 1 and 2.

22. The method of claim 18, wherein the at least 10 genes are listed in Tables 3 and 4.

23. The method of claim 18, wherein the at least 10 genes are listed in Tables 5 and 6.

24. The method of claim 18, wherein the at least 10 genes are listed in Tables 7 and 8.

25. The method of any one of claims 21 to 24, wherein the at least 10 genes have a fold change magnitude of at least about 1.5 (up) or 0.8 (down) in Table 2, 4, 6, or 8.

26. A method for evaluating the sensitivity of a tumor to one or a combination of therapeutic agents, comprising:
preparing a gene expression profile for a tumor specimen according to any one of claims 1 to 25; and
determining the presence of at least one gene expression signature indicative of drug-sensitivity or drug-resistance, thereby classifying the profile as a drug-sensitive or drug-resistant profile.

27. The method of claim 26, wherein the gene expression signature comprises threshold gene expression values indicative of drug sensitivity and/or drug resistance.

28. The method of claim 26 or 27, wherein the gene expression signature comprises Mean gene expression levels indicative of drug sensitivity and/or drug resistance.

29. The method of any one of claims 26 to 28, wherein the gene expression signature is predictive of efficacy for one or more of treatment with TFAC, EC, AC or ACT.

30. The method of any one of claims 26 to 29, wherein the gene expression profile is classified by Principal Components Analysis, Naïve Bayes, Support Vector Machines, Nearest Neighbors, Decision Trees, Logistic, Artificial Neural Networks, and Rule-based schemes.

31. The method of any one of claims 26 to 30, wherein the gene expression signature is predictive of survival, pathological complete response (pCR), reduction in tumor size, or duration of progression free interval upon treatment with a chemotherapeutic agent or combination.

32. The method of any one of claims 26 to 31, further comprising, conducting an in vitro chemoresponse assay with cultured cells derived from the patient tumor specimen.

33. A computer system for performing the method of any one of claims 1-32.

34. A probe array or probe set for performing the method of any one of claims 1-33.
